(19) United States
(12) Patent Application Publication
(10) Pub. No.: US 2008/0194711 A1
(43) Pub. Date: Aug. 14, 2008

(54) Cx3Cr1 As A Marker Which Correlates With Both Disease And Disease Activity In Multiple Sclerosis Patients

(76) Inventors: Frauke Zipp-Nitsch, Berlin (DE); Carmen Infante-Duarte, Berlin (DE); Claus-Stefen Stuerzebecher, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/814,229

(22) PCT Filed: Jan. 19, 2006
(86) PCT No.: PCT/EP2006/000466

§ 371 Date: Apr. 2, 2008

(30) Foreign Application Priority Data
Jan. 19, 2005 (EP) 05001062.8

(51) Int. Cl. C12Q 1/68; A61P 37/00; A61K 45/00

(52) U.S. Cl. 514/789; 435/6

(57) ABSTRACT

Multiple sclerosis (MS) is an autoimmune disease of the central nervous system (CNS) characterized by an enormous variability in its clinical presentation and course, in which clear diagnostic parameters are lacking. Here, the inventors performed an RNA screen, which indicated a role for the chemokine receptor CX₃CR1 as diagnostic marker Gene expression and flow cytometric analyses demonstrated a significantly lower expression of CX₃CR1 in MS patients compared to healthy individuals. Importantly, the inventors also found a correlation between disease activity and frequency of CX₃CR1⁺ NK cells. These findings emphasise the involvement of NK cells in the development and course of MS and provide evidence for CX₃CR1 expression to be a marker for MS patients and disease activity.

The present invention relates to the chemokine receptor CX₃CR1 as diagnostic marker in multiple sclerosis (MS). Gene expression and flow cytometric analyses demonstrated a significantly lower expression of CX₃CR1 in MS patients compared to healthy individuals. Importantly, the inventors also found a correlation between disease activity and frequency of CX₃CR1⁺NK cells. These findings emphasise the involvement of NK cells in the development and course of MS and provide evidence for CX₃CR1 expression to be a marker for MS patients and disease activity. Consequently, in vitro methods for the diagnosis of multiple sclerosis (MS) in a patient based on CX₃CR1 and aspect related thereto are provided.

### DESCRIPTION

Multiple sclerosis (MS) is an inflammatory demyelinating disease of the central nervous system (CNS) that represents a major cause of severe disability in young adults in western countries. Although MS is considered to be an autoimmune disease of unclear aetiology, it is assumed that disease susceptibility is genetically determined while the onset is governed by environmental factors (Noseworthy J H, Lucchinetti C, Rodriguez M, Weinshenker B G. Multiple sclerosis. N Engl J Med. 2000; 343:938-952; O'Connor P. Key issues in the diagnosis and treatment of multiple sclerosis. An overview. Neurology. 2002; 59:S1-33). The polygenic aetiology of MS and its multiple possible interactions with environmental factors determine the phenotypical heterogeneity of the disease, characterised by an enormous variability in the clinical presentation and course of the illness that greatly complicates the physician's ability to diagnose and make prognoses in MS. At the time of its onset, MS can be clinically classified as relapsing-remitting MS (RRMS), in which acute attacks are followed by complete or partial recovery to the pre-existing stage of disability, and primary progressive MS (PPMS), characterised by disease progression from onset, which can occasionally be interrupted by plateaus (Noseworthy J H, Lucchinetti C, Rodriguez M, Weinshenker B G. Multiple sclerosis. N Engl J Med. 2000; 343:938-952; O'Connor P. Key issues in the diagnosis and treatment of multiple sclerosis. An overview. Neurology. 2002; 59:S1-33; Hafler D A. Multiple sclerosis. J Clin Invest. 2004; 113:788-794). Thus, within both RRMS and PPMS clinical phenotypes, stable as well as acute phases of the disease can occur.

In the attempt to identify a gene expression profile characteristic for MS, several expression studies have been published in the last 3 years. In all these studies, the authors investigated the gene expression pattern of peripheral blood mononuclear cells (PBMC) from RRMS patients, generally using cDNA microarrays (Ramanathan M, Weinstock-Guttman B, Nguyen L T, et al. In vivo gene expression revealed by cDNA arrays: the pattern in relapsing-remitting multiple sclerosis patients compared with normal subjects. J Neuroimmunol. 2001; 116:213-219; Maas K, Chan S, Parker J, et al. Cutting edge: molecular portrait of human autoimmune disease. J Immunol. 2002; 169:5-9; Bomprezzi R, Ringner M, Kim S, et al. Gene expression profile in multiple sclerosis patients and healthy controls: identifying pathways relevant to disease. Hum Mol Genet. 2003; 12:2191-2199) or DNA microarrays (Achiron A, Gurevich M, Friedman N, Kaminski N, Mandel M. Blood transcriptional signatures of multiple sclerosis: unique gene expression of disease activity. Ann Neurol. 2004; 55:410-417; Iglesias A H, Camelo S, Hwang D, Villanueva R, Stephanopoulos G, Dangond F. Microarray detection of E2F pathway activation and other targets in multiple sclerosis peripheral blood mononuclear cells. J Neuroimmunol. 2004; 150:163-177).

Despite the above-mentioned attempts to provide markers that aid in the diagnosis, prognosis, and/or monitoring of MS, the set of presently available markers still is insufficient.

It is therefore an object of the present invention, to provide further markers that not only define MS but are also helpful in the prognosis, and/or monitoring of MS, in particular in RRMS or PPMS. It is a further object of the present invention, to provide respective methods for a diagnosis, prognosis, and/or monitoring of MS and treatment thereof, based on said markers. It is a further object of the present invention, provide respective methods for distinguishing between the patient's status of stable disease (remission or recovery) and active disease (relapse) states in RRMS or PPMS.

In a first aspect of the present invention, this object is solved by an in vitro method for the diagnosis of multiple sclerosis (MS) in a patient, comprising a) providing a biological sample from the patient to be diagnosed, b) providing a biological sample from a healthy donor or a reference value indicative for a healthy donor, and c) determining the level of expression of CX3CR1 gene or protein expression in said samples, wherein a reduction of the relative expression of more than 30%, preferably 40%, compared to said healthy donor is indicative of MS in said patient. The reference values might be taken from the literature (e.g. a respective chart) and/or earlier experiments or can be determined in the same and/or a parallel respective experiment or examination regarding CX3CR1 gene or protein expression.

For the purpose of identifying markers, facilitating not only the definition of MS but also of its courses, the inventors expanded the investigation and performed a functional genomic analysis of patients suffering either from RRMS or PPMS, as well as of healthy individuals. The inventors used high density oligonucleotide-based DNA microarrays, considered to be more reproducible than cDNA microarrays (Li J, Pankratz M, Johnson J A. Differential gene expression patterns revealed by oligonucleotide versus long cDNA arrays. Toxicol Sci. 2002; 69:383-390), and analysed the expression of 12,000 genes in the three different cohorts, RRMS and PPMS patients, and HD. Interestingly, the inventors did not detect significant gene regulation differences in RRMS vs. PPMS patients, but were able to identify 6 genes that were highly significantly and differentially regulated in both groups of patients compared to control individuals. One of these genes encodes the chemokine receptor CX₃CR1 (chemokine (C-X3-C motif) receptor 1, also known as G-protein coupled receptor 13, fractalkine receptor CX3CR1, and chemokine (C-C) receptor-like 1), which showed a decreased expression in both PPMS and RRMS patients when compared to healthy controls.

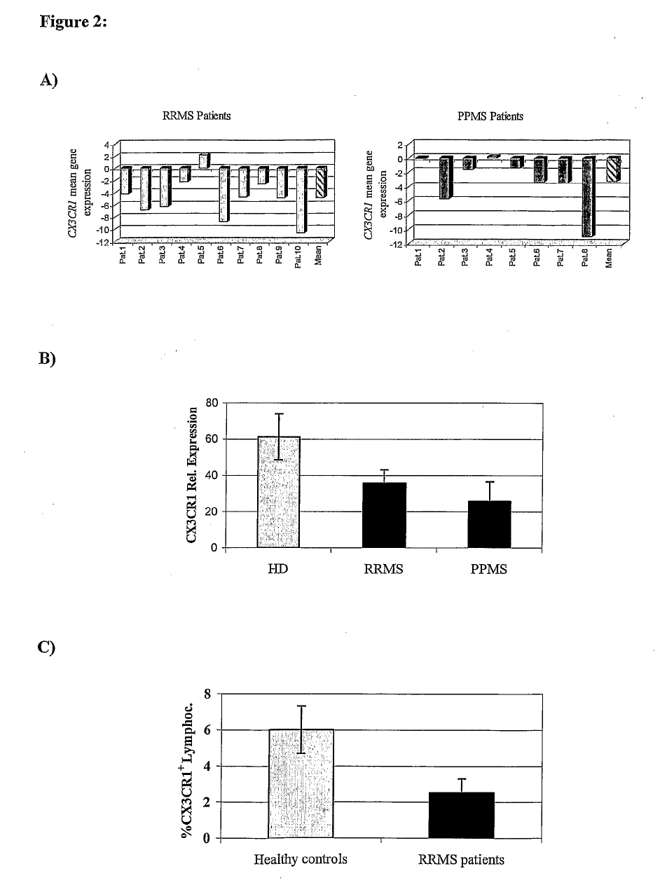

In one preferred embodiment thereof, the in vitro method for the diagnosis of multiple sclerosis in a patient as described above is characterised in that the relative expression of the CX3CR1 gene is reduced by at least 2-fold, and preferably 3-fold. This expression usually is obtained by using a microarray setting (see also examples, below). Preferably, the relative expression of the CX3CR1 gene is reduced by at least 4-fold, most preferred is a reduction of at least 4.3-fold in RRMS and 3,7-fold in PPMS patients (see FIG. 2).

In yet another preferred embodiment thereof, the in vitro method for the diagnosis of multiple sclerosis in a patient as described above is characterised in that said the biological samples are enriched for peripheral mononuclear cells (PBMC). Such enriched fractions therefore contain higher numbers of particular groups of cell types, such as monocytes, T-lymphocytes, B-lymphocytes, and in particular dendritic or natural killer (NK) cells.

In yet another preferred embodiment thereof, the in vitro method for the diagnosis of multiple sclerosis in a patient as described above is characterised in that said patient suffers from relapsing-remitting MS (RRMS), in which acute attacks are followed by complete or partial recovery to the pre-existing stage of disability; or primary progressive MS (PPMS) characterised by disease progression from onset, which can occasionally be interrupted by plateaus. Thus, within both RRMS and PPMS clinical phenotypes, stable as well as acute phases of the disease can occur.

RRMS and PPMS clinical phenotypes are further described in Rovaris M, Filippi M. (MR-based technology for in vivo detection, characterization, and quantification of pathology of relapsing-remitting multiple sclerosis. J Rehabil Res Dev. 2002 March-April; 39(2):243-59) and Ukkonen M, Elovaara I, Dastidar P, Tamiela T L. (Urodynamic findings in primary progressive multiple sclerosis are associated with increased volumes of plaques and atrophy in the central nervous system. Acta Neurol Scand. 2004 February; 109(2):100-5) as well as Boylan M T, Crockard A D, McDonnell G V, McMillan S A, Hawkins S A. (Serum and cerebrospinal fluid soluble Fas levels in clinical subgroups of multiple sclerosis. Immunol Lett. 2001 Oct. 1; 78(3):183-7) and Kurtzke J F. (Clinical definition for multiple sclerosis treatment trials. Ann Neurol. 1994; 36 Suppl:S73-9).

In yet another preferred embodiment thereof, the in vitro method for the diagnosis of multiple sclerosis in a patient as described above is characterised in that the gene expression is reduced by at least 30% and at least 50% in patients suffering from RRMS and PPMS, respectively. This expression usually is obtained by using an rtPCR (reverse transcriptase polymerase chain reaction) or TaqMan® setting (see also examples, below). Preferably, the gene expression of is reduced by at least 35% and at least 55% in patients suffering from RRMS and PPMS, respectively. Most preferred are at least 40% and at least 57% in patients suffering from RRMS and PPMS, respectively.

The marker according to the present invention surprisingly allows for an improved monitoring of the progression of the disease, e.g. by the attending physician. In the context of the present invention, a “stable” disease phase of MS, and particularly in case of the clinical phenotypes RRMS and PPMS, can be defined as a remission or recovery as based on the diagnostic parameters as determined for the patient suffering from MS (see respective publications as cited herein, e.g. as above), i.e. a complete or partial recovery to the pre-existing stage of disability. In addition, a stable disease phase would also encompass a steady state of the diagnostic parameters, i.e. a stop or pausing of the progression of the disease, whether occurring naturally or through suitable treatment of the patient. In contrast to a stable disease phase, an “acute” disease phase of MS, and particularly in case of the clinical phenotypes RRMS and PPMS is defined herein as an acute (ongoing) worsening, for example in the form of a relapse, of the disease as determined based on the diagnostic parameters as measured for the patient suffering from MS (see respective publications as cited herein, e.g. as above). One example of said parameters would be the occurrence of lesions (whether they are phenotypically visible or not) that form during the progression of the disease.

In another particularly preferred embodiment thereof, the in vitro method for the diagnosis of an acute and/or stable multiple sclerosis in a patient according to the present invention comprises the steps of a) providing a biological sample from the patient to be diagnosed that is enriched for NK cells, and b) determining the CX3CR1 protein expression in said sample, wherein an expression of CX3CR1 protein in more than 15% of the NK cells is indicative of acute (active) MS in said patient. Preferably, said patients are characterised in that they are diagnosed as suffering from RRMS, either in a stable disease (remission or recovery) or active disease (relapse) state phase.

In yet another preferred embodiment thereof, the in vitro method for the diagnosis of multiple sclerosis in a patient as described above comprises determining said CX3CR1 gene or protein expression comprises microarray analysis, real-time PCR and/or flow cytometry analysis. Other methods for determining gene or protein expression are known to the person of skill in the art and can be taken from the respective literature, these can also be applied, such as antigen-based tests.

In another particularly preferred embodiment thereof, the in vitro method for the diagnosis of multiple sclerosis in a patient as described above said biological sample is whole blood or serum and said CX3CR1 protein expression analysis comprises flow cytometry analysis. Preferably, such flow cytometry analysis comprises staining with anti-CD3 FITC/anti-CD56CD16 PE, anti-CD3/anti-CD4 or anti-CD3/anti-CD8.

In yet another preferred embodiment thereof, the in vitro method for the diagnosis of multiple sclerosis in a patient as described above is characterised in that said method is performed at least one to four times a year. Of course, the actual scheme of performing said method will be decided by the attending physician, for example based on the particular status of the patient that will be diagnosed and/or monitored. The frequency can be increased or decreased, depending again from the particular status of the patient that will be diagnosed and/or monitored.

Another preferred aspect of the present invention is related to a method of treatment for multiple sclerosis in a patient, comprising the steps of a) performing the method as described above, and b) providing a suitable medication to a patient in need thereof. Such medication can comprise all suitable therapies of MS known to the person of skill, such as interferon, statins, immunomodulators such as mitoxantrone, steroids, neutralising antibodies, and/or bee venom and combinations thereof. In one embodiment of said method of treatment for multiple sclerosis in a patient as described above, the said patient suffers from acute RRMS or PPMS.

Another preferred aspect of the present invention is related to a kit, comprising suitable materials and compounds for performing the method as described above. Said materials are generally known to the person of skill in the art and can be selected from chemicals, buffers, disposable test devices, foil packs, instructions for using said test kit, buffers, antibodies, colour charts, reference blood or serum samples from healthy donors, and plastic materials such as dishes and tubes. More preferred is a kit as described above, wherein said kit comprises materials and compounds suitable for a point-of-care analysis. Point-of-care analysis is normally defined as testing that is performed outside the physical facilities of the clinical laboratory, in proximity to the patient on whom the testing is performed. The central feature of POC testing is that it does not require permanent, dedicated space.

Another preferred aspect of the present invention finally relates to the use of CX3CR1 expression for distinguishing between healthy and MS patients and/or for distinguishing between acute and stable MS disease phase, wherein the same considerations apply as described above for the other aspects of the present invention. In particular, the in vitro methods for the diagnosis and/or monitoring of multiple sclerosis in a patient as described herein are characterised in that said methods are performed at least one to four times a year. Of course, the actual scheme of performing said method will be decided by the attending physician, for example based on the particular status of the patient that will be diagnosed and/or monitored. The frequency can be increased or decreased, depending again from the particular status of the patient that will be diagnosed and/or monitored.

The marker according to the present invention allows for an improved monitoring of the progression of the disease by the attending physician. Importantly, a treatment can be initiated even if the acute pathological processes are not yet directly visible by external phenomena. For a diagnosis, the expression of the marker can easily be measured based on the blood or serum of the patient and FACS-determination. Until now, the monitoring of the disease as well as the diagnosis of lesions that are not phenotypically visible can only be achieved by very expensive MRT examinations. Thus, a high demand exists for a cheap and gentle but effective examination method. The present invention fulfils these demands.

CX₃CR1 is the unique receptor for fractalkine (CX₃CL1), a chemokine that exists in soluble and surface-bound form (Bazan J F, Bacon K B, Hardiman G, et al. A new class of membrane-bound chemokine with a CX3C motif. Nature. 1997; 385:640-644), and mediated both chemotaxis and adhesion of leukocytes (Fong A M, Robinson L A, Steeber D A, et al. Fractalkine and CX3CR1 mediate a novel mechanism of leukocyte capture, film adhesion, and activation under physiologic flow. J Exp Med. 1998; 188:1413-1419; Haskell C A, Cleary M D, Charo I F. Molecular uncoupling of fractalkine-mediated cell adhesion and signal transduction. Rapid flow arrest of CX3CR1-expressing cells is independent of G-protein activation. J Biol Chem. 1999; 274:10053-10058). CX₃CR1 is expressed on monocytes, NK cells and activated T cells, preferentially Th1-like cells (Fraticelli P, Sironi M, Bianchi G, et al. Fractalkine (CX3CL1) as an amplification circuit of polarized Th1 responses. J Clin Invest. 2001; 107:1173-1181) but not on B cells (Nishimura M, Umehara H, Nakayama T, et al. Dual functions of fractalkine/CX3C ligand 1 in trafficking of perforin+/granzyme B+ cytotoxic effector lymphocytes that are defined by CX3CR1 expression. J Immunol. 2002; 168:6173-6180). Moreover it is expressed on those cytotoxic lymphocytes containing high levels of intracellular perform and granzyme B, suggesting an involvement of the receptor in migration of cytotoxic effector lymphocytes (Nishimura M, Umehara H, Nakayama T, et al. Dual functions of fractalkine/CX3C ligand 1 in trafficking of perforin+/granzyme B+ cytotoxic effector lymphocytes that are defined by CX3CR1 expression. J Immunol. 2002; 168:6173-6180) and pro-inflammatory Th cells (Fraticelli P, Sironi M, Bianchi G, et al. Fractalkine (CX3CL1) as an amplification circuit of polarized Th1 responses. J Clin Invest. 2001; 107:1173-1181). Since these cells are considered to be of particular importance in the pathogenesis of MS, the inventors focused on the investigation of the role of chemokine receptor CX₃CR1 in MS patients.

Sunnemark et al. (in Sunnemark D, Eltayeb S, Wallstrom E, Appelsved L, Malmberg A, Lassmann H, Ericsson-Dahlstrand A, Piehl F, Olsson T., Differential expression of the chemokine receptors CX3CR1 and CCR1 by microglia and macrophages in myelin-oligodendrocyte-glycoprotein-induced experimental autoimmune encephalomyelitis. Brain Pathol. 2003 October; 13(4):617-29) describe a study on the in vivo expression of receptors for the chemokines CCL3/CCL5/CCL7 (MIP-1alpha/RANTES/MCP-3) and CX3CL1 (fractalkine), CCR1 and CX3CR1, respectively, in rat myelin oligodendrocyte glycoprotein-induced experimental autoimmune encephalomyelitis. Combined in situ hybridization and immunohistochemistry demonstrated intensely upregulated CCR1 mRNA expression in early, actively demyelinating plaques, whereas CX3CR1 displayed a more generalized expression pattern. They suggest a differential receptor expression between microglia and monocyte-derived macrophages and that mainly the latter cell type is responsible for active demyelination. This would have great relevance for the possibility of therapeutic intervention in demyelinating diseases such as multiple sclerosis, for example by targeting signalling events leading to monocyte recruitment. Sunnemark et al. teach away from using CX3CR1 as a diagnostic marker, due to its more generalized expression pattern.

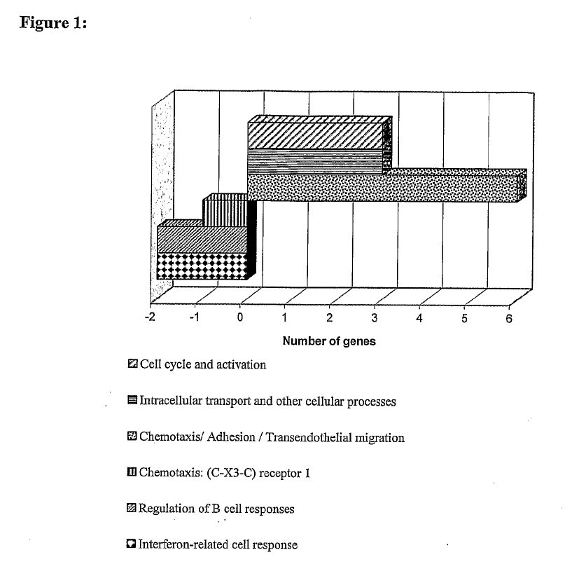

Employing oligonucleotide arrays, the inventors identified 17 genes that were differentially expressed in PBMC of MS patients compared to healthy individuals. Sixteen of them were specifically regulated in RRMS and one was differentially expressed only in PPMS patients compared to HD. Genes with increased expression were involved in: (1) cell cycle and activation, (2) chemotaxis, adhesion and transendothelial migration, (3) intracellular transport mechanisms and other cellular processes. Genes with decreased expression, on the other hand, were involved in the response of monocytes to interferons and in the regulation of B cell response (FIG. 1). By comparing females and males in the groups of patients, the inventors demonstrated that the observed gene expression profile in MS patients was not gender-dependent (data not shown). Of the 16 genes differentially regulated in RRMS patients, six were similarly expressed in PPMS.

Decreased CX₃CR1 gene and protein expression in MS patients—The data obtained using microarray analysis indicates that expression of the chemokine receptor CX₃CR1 is decreased in PBMC of both RRMS and PPMS when compared to HD. The relative expression of the receptor (related to HD) was −4.9 in RRMS and −3.3 in PPMS (FIG. 2A). To confirm these results the inventors examined gene expression of CX₃CR1 in expanded patient and HD cohorts using real-time rtPCR (HD n=28; RRMS patients n=25; and PPMS patients n=20) and protein expression using flow cytometry analysis in another 19 RRMS patients vs. 19 HD (included in Table 1C). The data plotted in FIGS. 2B and C (mean relative expression +SEM) confirm that MS patients show a reduced CX₃CR1 gene (FIG. 2B) and protein expression (FIG. 2C) compared to control individuals. Gene expression was reduced by 40% and 57% in PBMC from RRMS and PPMS patients respectively when compared to HD. For the protein analysis, the inventors focused on patients with RRMS in who the inventors found reduced expression of CX₃CR1 by 58% on lymphocytes (FIG. 2C).

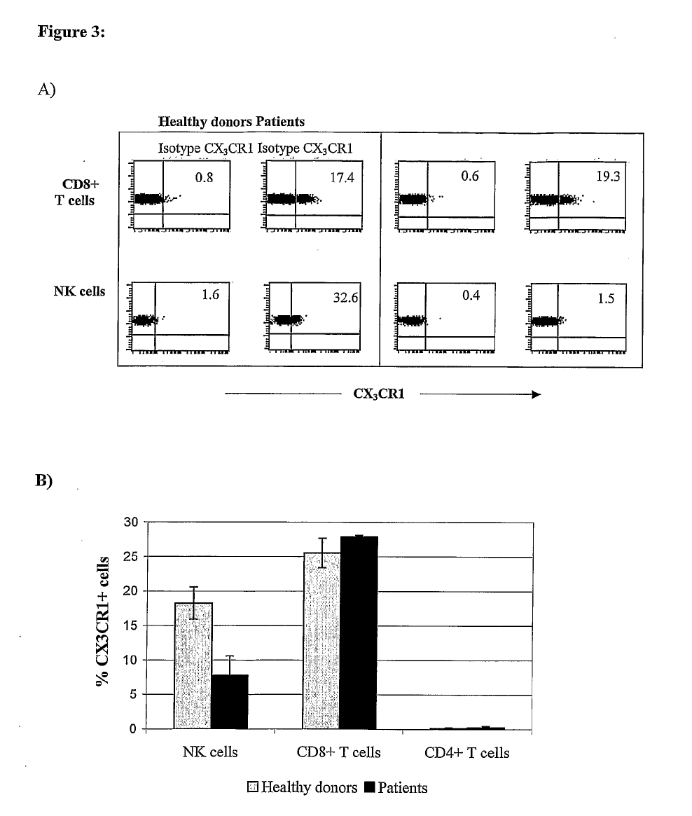

Reduced expression of CX₃CR1 on NK cells, but not on cytotoxic T cells of MS patients—Using multiparameter flow cytometry analysis, the inventors studied whether the expression of CX₃CR1 was downregulated in all lymphocytes in MS patients or whether it was characteristic of a particular lymphocyte subpopulation. Analyzing the expression of CX₃CR1 on NK-cells, CD4⁺ T cells and CD8⁺ cytotoxic T cells, the inventors showed the reduced expression only on NK cells and not on CD4⁺ or CD8⁺ T cells (FIGS. 3A and 3B). The receptor expression on CD4⁺ T cells was almost undetectable in both patients and healthy controls (FIG. 3B).

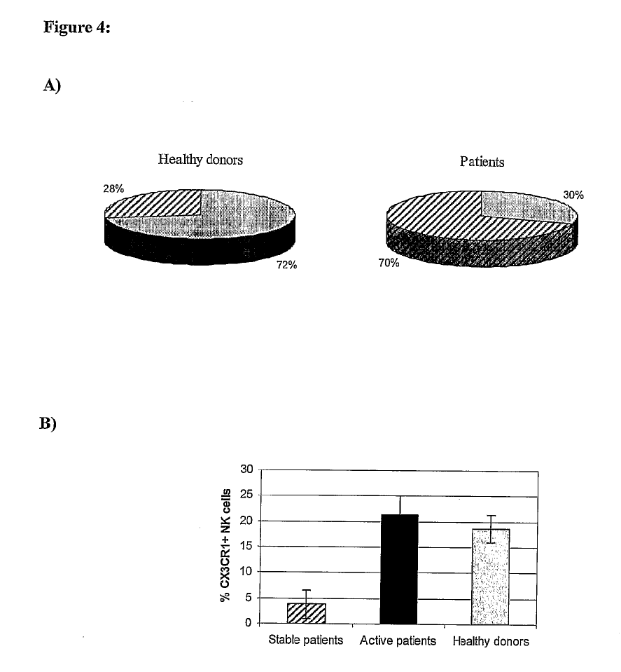

Correlation between clinical disease manifestations and CX₃CR1 expression—A detailed analysis of CX₃CR1 expression in RRMS patients and HD indicated that about 72% of the healthy individuals expressed an usual amount of CXCR1 on NK cells (shaded sector of the pie chart) while 28% of them expressed low or undetectable levels of the receptor (hatched sector of the chart). The patient profile, on the other hand, was exactly the opposite, with approximately 70% of the patients showing little or no receptor expression on NK cells, while the other 30% presented an expression comparable with that of the healthy population (FIG. 4A). The inventors observed that the majority of this 30% of patients showing “normal” receptor expression shared common clinical characteristics and were in an active disease phase, while the patients with low expression were in a stable condition. The next aim was thus to establish whether or not receptor expression correlated in effect with clinical manifestations. Therefore, the inventors extended the analysis of CX₃CR1 expression on NK cells to additional healthy individuals and well-characterized RRMS patients. The clinical characteristics of the patients and the frequency of CX₃CR1⁺ NK-cells are summarized in Table 2. These verified that the majority (92%) of patients with stable disease presented a very low expression of CX₃CR1 on NK cells while the majority of patients (89%) suffering from acute relapses or with gadolinium (Gd) enhancing magnetic resonance (MR) lesions showed increased receptor expression.

The patient data, together with the mean expression of the receptor in healthy individuals, are plotted in FIG. 4B. This figure shows that the frequency of CX₃CR1⁺NK cells in patients with active disease was comparable with the frequency of those cells in healthy controls and four times higher than in stable patients (FIG. 4B).

The inventors used large-scale gene expression analysis and quantitative real-time rtPCR to identify marker genes for both RRMS and PPMS patients. In line with other expression analyses performed with cDNA microarrays the present findings emphasize the role of immune and cell cycle related genes in MS (Ramanathan M, Weinstock-Guttman B, Nguyen L T, et al. In vivo gene expression revealed by cDNA arrays: the pattern in relapsing-remitting multiple sclerosis patients compared with normal subjects. J Neuroimmunol. 2001; 116:213-219; Maas K, Chan S, Parker J, et al. Cutting edge: molecular portrait of human autoimmune disease. J Immunol. 2002; 169:5-9; Bomprezzi R, Ringner M, Kim S, et al. Gene expression profile in multiple sclerosis patients and healthy controls: identifying pathways relevant to disease. Hum Mol Genet. 2003; 12:2191-2199) and, moreover, highlight the involvement of transmigration related mechanisms in the disease (FIG. 1). One such gene involved in chemoattractant processes was that for CX₃CR1. CX₃CR1 is expressed on cytotoxic NK and CD8⁺ T cells and on CD4⁺Th1 cells. Its ligand, fractalkine (CX₃CL1), is produced by, among others, endothelial cells. CX₃CL1 expression is increased under pro-inflammatory conditions, such as the presence of IFN-gamma, which promotes transmigration of CX₃CR1⁺ cells in inflammation (Yoneda O, Imai T, Nishimura M, et al. Membrane-bound form of fractalkine induces IFN-gamma production by NK cells. Eur J Immunol. 2003; 33:53-58).

Here the inventors show that PBMC from MS patients contain a decreased frequency of CX₃CR1-positive mononuclear cells when compared with those of healthy individuals. This diminished expression was observed on both RNA and protein level, affecting exclusively the NK cells and not the cytotoxic T cell population. CX₃CR1⁺ NK (cells express high levels of CD57, CD11b, and CD11a and have large amounts of intracellular cytotoxic granules containing perforin and granzyme (Nishimura M, Umehara H, Nakayama T, et al. Dual functions of fractalkine/CX3C ligand 1 in trafficking of perforin+/granzyme Bu cytotoxic effector lymphocytes that are defined by CX3CR1 expression. J Immunol 2002; 168:6173-6180). This indicates that these cells represent a cytotoxic effector population which is capable of migrating to inflamed tissues and is involved in the pathology of different disorders, such as atherosclerosis, renal diseases or rheumatoid arthritis (Umehara H, Bloom E T, Okazaki T, Nagano Y, Yoshie O, Imai T. Fractalkine in vascular biology: from basic research to clinical disease. Arterioscler Thromb Vasc Biol. 2004; 24:34-40).

In MS, the role of the CX₃CL1/CX₃CR1 pathway is not known. It has been reported that MS patients suffering from relapses showed a markedly increased level of CX₃CL1 in the serum when compared to patients with other inflammatory diseases of the CNS and other patients with noninflammatory disorders of the CNS (Kastenbauer S, Koedel U, Wick M, Kieseier B C, Hartung H P, Pfister H W. CSF and serum levels of soluble fractalkine (CX3CL1) in inflammatory diseases of the nervous system. J Neuroimmunol. 2003; 137:210-217). In the CNS, CX₃CL1 is expressed by neurons and astrocytes, while the receptor is expressed on neurons, microglia and astrocytes (Hatori K, Nagai A, Heisel R, Ryu J K, Kim S U. Fractalkine and fractalkine receptors in human neurons and glial cells. J Neurosci Res. 2002; 69:418-426. Hulshof S, van Haastert E S, Kuipers H F, et al. CX3CL1 and CX3CR1 expression in human brain tissue: noninflammatory control versus multiple sclerosis. J Neuropathol Exp Neurol. 2003; 62:899-907). Although CX₃CL1 can be upregulated under pro-inflammatory conditions, induction of acute inflammation in rodent CNS did not affect the brain expression of ligand and receptor (Hughes P M, Botham M S, Frentzel S, Mir A, Perry V H. Expression of fractalkine (CX3CL1) and its receptor, CX3CR1, during acute and chronic inflammation in the rodent CNS. Glia. 2002; 37:314-327), and so far no differences in CX₃CL1 expression in brain tissue have been detected between control patients and MS patients (Hulshof S, van Haastert E S, Kuipers H F, et al. CX3CL1 and CX3CR1 expression in human brain tissue: noninflammatory control versus multiple sclerosis. J Neuropathol Exp Neurol. 2003; 62:899-907).

CX₃CR1⁺ NK (cells are exclusively naturally cytotoxic CD56(dim) NK cells, and do not represent a population with immunomodulatory capacity (Cooper M A, Fehniger T A, Caligiuri M A. The biology of human natural killer-cell subsets. Trends Immunol. 2001; 22:633-640). The reduced frequency of CX₃CR1⁺NK cells demonstrated in the present invention might imply the presence of a defective cytotoxic effector NK cell population in MS patients confirming several reports of deficient NK cell activity in MS (Benczur M, Petranyl G G, Palffy G, et al. Dysfunction of natural killer cells in multiple sclerosis: a possible pathogenetic factor. Clin Exp Immunol. 1980; 39:657-662; Neighbour P A, Grayzel A I, Miller A E. Endogenous and interferon-augmented natural killer cell activity of human peripheral blood mononuclear cells in vitro. Studies of patients with multiple sclerosis, systemic lupus erythematosus or rheumatoid arthritis. Clin Exp Immunol. 1982; 49:11-21; Hirsch R L, Johnson K P. Natural killer cell activity in multiple sclerosis patients treated with recombinant interferon-alpha 2. Clin Immunol Immunopathol. 1985; 37:236-244; Vranes Z, Poljakovic Z, Marusic M. Natural killer cell number and activity in multiple sclerosis. J Neurol Sci. 1989; 94: 115-123; Kastrukoff L F, Morgan N G, Zecchini D, et al. A role for natural killer cells in the immunopathogenesis of multiple sclerosis. J Neuroimmunol. 1998; 86:123-133; Baxter A G, Smyth M J. The role of NK cells in autoimmune disease. Autoimmunity. 2002; 35:1-14). Furthermore, Kastruhoff et al. have reported on an increased NK cells activity during remissions, while relapses were preceded by reduced functional activity (Kastrukoff L F, Lau A, Wee R, Zecchini D, White R, Paty D W. Clinical relapses of multiple sclerosis are associated with ‘novel’ valleys in natural killer cell functional activity. J Neuroimmunol. 2003; 145:103-114). NK cells appear to be essential for regulating the immune response and the development of autoimmune processes (Horwitz D A, Gray J D, Ohtsula K, Hirokawa M, Takahashi T. The immunoregulatory effects of NK cells: the role of TGF-beta and implications for autoimmunity. Immunol Today. 1997; 18:538-542; Heusel J W, Ballas Z K. Natural killer cells: emerging concepts in immunity to infection and implications for assessment of immunodeficiency. Curr Opin Pediatr. 2003; 15:586-593). Their immunoregulatory properties were demonstrated in the mouse model of MS, experimental autoimmune encephalomyelitis (EAE), in which NK cells are probably involved in protection against autoimmunity (Zhang B, Yamamura T, Kondo T, Fujiwara M, Tabira T. Regulation of experimental autoimmune encephalomyelitis by natural killer (NK) cells. J Exp Med. 1997; 186:1677-1687).

Yet, in rheumatoid arthritis patients an increased CX₃CR1 expression on CD8⁺ T cells were found (Ruth J H, Rottman J B, Katschke K J, Jr., et al. Selective lymphocyte chemokine receptor expression in the rheumatoid joint. Arthritis Rheum. 2001; 44:2750-2760; Nanki T, Imai T, Nagasaka K, et al. Migration of CX3CR1-positive T cells producing type 1 cytokines and cytotoxic molecules into the synovium of patients with rheumatoid arthritis. Arthritis Rheum. 2002; 46:2878-2883), while systemic lupus erythematosus and psoriasis patients show normal expression of the receptor on T lymphocytes and NK cells respectively (Amoura Z, Combadiere C, Faure S, et al. Roles of CCR2 and CXCR3 in the T cell-mediated response occurring during lupus flares. Arthritis Rheum. 2003; 48:3487-3496; Echigo T, Hasegawa M, Shimada Y, Takehara K, Sato S. Expression of fractalkine and its receptor, CX3CR1, in atopic dermatitis: possible contribution to skin inflammation. J Allergy Clin Immunol. 2004; 113:940-948). These facts indicate that a differential CX₃CR1 expression on lymphocytes is not a phenomenon accompanying inflammatory autoimmune disorders in general but rather specific for MS. Whereas CX₃CR1 expression on NK cells was overall decreased in stable RRMS, the frequency of CX₃CR1⁺ NK cells was even increased during relapses or active phases compared to that detected in healthy individuals. Regular CX₃CR1 expression in patients clearly correlated with acute relapses and/or with the presence of gadolinium enhancing magnetic resonance lesions (Table 2 and FIG. 4).

The precise roles of NK cell subsets in MS patients are not elucidated so far. Recently Takahashi et al. have shown that NK cells involved in clinical remission are CD95-positive, produce type 2 cytokines and are able to inhibit autoreactive cells (Takahashi K, Miyake S, Kondo T, et al. Natural killer type 2 bias in remission of multiple sclerosis. J Clin Invest. 2001; 107:R23-29; Takahashi K, Aranami T, Endoh M, Miyake S, Yamamura T. The regulatory role of natural killer cells in multiple sclerosis. Brain. 2004; 127:1917-1927). In contrast, CX₃CR1⁺ NK cells are known to produce type 1 cytokines. Thus, the here reported increase of CX₃CR1⁺ NK cells during relapses may indicate a critical role of this pro-inflammatory subset of the NK cells for disease exacerbation.

In summary, the present invention demonstrated that CX₃CR1 expression on NK cells from MS patients, in particular RRMS patients, is dependent on disease activity and is particularly reduced in patients with a stable RRMS disease course. The results confirm and highlight the important role of innate immunity, particularly that of NK cells, in MS and, more importantly, propose the quantification of CX₃CR1⁺ NK (cells as a novel immunological parameter, not only for monitoring NK cell activity and facilitating MS diagnosis, but also as a reliable tool for monitoring disease course and supporting disease prognosis.

The features disclosed in the present description, in the claims and/or in the accompanying drawings may, both separately and in any combination thereof, be material for realising the invention in diverse forms thereof. The present invention will now be further described based on the following examples and the accompanying Figures, wherein

FIG. 1 shows the functional classification of genes differentially expressed in multiple sclerosis. Genes were chosen according to the selection strategies explained in Methods. Using an expression cutoff of greater than 2, a total of 17 genes was identified that were regulated differentially in MS patients. The figure shows the functional grouping of the identified genes with increased expression (to the right) and with decreased expression (to the left) in MS patients vs. HD. The number of genes for each group is displayed.

FIG. 2 shows CX₃CR1 expression in peripheral blood mononuclear cells from patients with MS. (A) The diagrams display the results of CX₃CR1 gene expression obtained from the microarray analysis for 10 RRMS (left graph) and 8 PPMS patients (right graph) respectively. For each patient, the mean expression of the 12 pairwise comparisons (the particular patient vs. the 12 healthy controls) is shown. The hatched bars in the right part of each diagram represent the mean expression of the 10 RRMS and 8 PPMS patients respectively. (B) Gene expression of CX₃CR1 was analyzed using real-time it PCR on PBMC from 28 healthy individual, 25 RRMS patients, and 20 PPMS patients. The data are normalized to housekeeping gene, 18S rRNA, and are expressed as the mean relative expression +SEM. Significant P values (<0.05) are indicated with asterisk (*) (C) CX₃CR1 protein expression was quantified by flow cytometric analyses performed on PBMC from 19 RRMS patients and 19 healthy individuals. The data are shown as the mean protein expression +SEM. The asterisk (*) indicates a significant p value (<0.05).

FIG. 3 shows a reduced expression of CX₃CR1 on NK cells but not on cytotoxic T lymphocytes in MS patients. CX₃CR1 protein expression was quantified by flow cytometric single-cell analyses performed on PBMC from RRMS patients and healthy individuals. (A) The dot plots demonstrate that CX₃CR1 expression on CD8⁺ T cells from MS patients and HD is similar, while the receptor expression on NK cells is reduced in MS patients. CX₃CR1 and the corresponding isotype staining are shown on the x-axis and staining for CD8 and NK cells on the y-axis. The results are representative for several staining analyses. (B) The figure shows the quantification of CX₃CR1 expression on NK cells, CD8⁺ T cells and CD4⁺ T cells in HD (bright bars) vs. MS patients (shaded bars). The y-axis shows the frequency of CX₃CR1-positive cells. The data are shown as the mean expression from 19 HD and 19 patients plus SEM. The asterisk (*) indicates a significant p value (<0.05).

FIG. 4 shows an expression of CX₃CR1 on NK cells from MS patients is increased during disease activity. Expression of CX₃CR1 on NK (cells from MS patients was quantified by flow cytometry analysis. (A) The pie chart shows the frequency of NK cells expressing little CX₃CR1 (hatched sector) versus the frequency of NK cells expressing large amount of CX₃CR1 (shaded sector). The diagram demonstrates that patients and HD present inverse distribution of the NK cell subgroups: contrary to the HD, 30% of the patients display a regular CX₃CR1⁺NK cells population while 70% of those shows a NIL cells population with reduced CX₃CR1 expression. (B) Patients displaying a regular CX₃CR1⁺NK cells population are those with active disease, i.e. suffering from acute relapses or with gadolinium enhancing MR lesions (dark bars), while the patients with stable disease course show decreased frequency of CX₃CR1⁺ NK cells (hatched bars). The y-axis shows the frequency of CX₃CR1-positive cells. The data are shown as the mean expression of the receptor from 28 HD, 13 stable patients and 9 patients with active disease, plus SEM. The asterisk (*) indicates a significant p value (<0.05).

### EXAMPLES

### Patients and Control Individuals

RNA levels were monitored in n=10 RRMS and n=8 PPMS patients, as well as in n=12 healthy control individuals, using oligonucleotide microarrays (Table 1A). To confirm the microarray data, an expanded cohort consisting of n=25 RRMS and n=20 PPMS patients, and n=28 healthy donors (HD) (Table 1B) was conducted to analyze the expression of CX₃CR1 using real-time rtPCR. Multiparameter cytometric analysis was performed in a cohort of 19 RRMS patients (15 of them were different from patients involved in Real-time rtPCR analysis) and 19 HD (14 of them were different from HD involved in Real-time rtPCR analysis) (Table 1C). Analysis of the correlation between disease activity and cytometric date was conducted in a cohort of 22 RRMS patients (seven of them were different from patients involved in the first cytometric analysis) and 28 HD (nine additional HD to them involved in the first cytometric analysis) (Table 1D).

Clinically definite MS patients with relapsing remitting (RRMS) or primary chronic progressive (PPMS) course (McDonald W I, Compston A, Edan G, et al. Recommended diagnostic criteria for multiple sclerosis: guidelines from the International Panel on the diagnosis of multiple sclerosis. Ann Neurol. 2001; 50:121-127) were enrolled at the Institute of Neuroimmunology of the University Hospital Charite. Patients (except Pat. 12) did not receive any immunomodulatory treatment and were not treated with steroids for at least 6 weeks before venipuncture. The disability score, as defined by the Expanded Disability Status Scale (EDSS) (Kurtzke J F. Rating neurologic impairment in multiple sclerosis: an expanded disability status scale (EDSS). Neurology. 1983; 33:1444-1452) was 0-4 in RRMS cases and 1.5-8 in PPMS patients. Patients' characteristics are given in Table 1.

Peripheral blood samples of patients and of randomly selected healthy donors were obtained with informed consent for use in this invention.

For RNA analysis, PBMC of patients and HD were isolated from fresh blood using Lymphoprep™ density gradient centrifugation (Nycomed Pharma, Roskilde, Denmark), and then cryopreserved in liquid nitrogen for later RNA preparation. RNA was extracted from about 6-7 million PBMC using RNeasy® Midi Kit (Qiagen, Santa Clarita, Calif., USA) according to the manufacturer's instructions.

### Microarray Analysis

10 μg of total RNA were converted into double-stranded cDNA using a modified oligo-dT primer including a 5′ T7 RNA polymerase promoter sequence and the Superscript Choice system for cDNA synthesis (LifeTechnologies, NY, USA). In vitro transcription was performed with T7 RNA polymerase (T7 Megascript kit, Ambion) and 0.5-1 μg of double-stranded cDNA template in the presence of a mixture of ATP, CTP, GTP, biotin-11-CTP, and biotin-16-UTP (ENZO Diagnostics, Farmingdale, N.Y., USA). 20 μg of cRNA were fragmented randomly by incubating in 40 mM Tris-acetate pH 8.1, 100 nm K⁺ acetate, and 30 mM Mg²⁺ acetate at 94° C. for 35 min. The Human Genome U95A arrays (Affymetrix, Santa Clara, Calif., USA) were hybridized, washed, and stained according to standard protocols (Mahadevappa M, Warrington J A. A high-density probe array sample preparation method using 10- to 100-fold fewer cells. Nat Biotechnol. 1999; 17:1134-1136), involving a three-step staining and amplification procedure with streptavidin-phycoerythrin (SAPE), a biotinylated anti-streptavidin antibody, and a final SAPE step. The fluorescence intensities for the individual oligonucleotide probes were determined using a confocal scanner from Affymetrix. To identify differentially regulated genes in the patient/HD groups, multiple pairwise comparisons were performed between the individual samples of two groups (RRMS vs. HD, PPMS vs. HD, PPMS vs. RRMS). P-values for groupwise comparisons were calculated using a commercially available algorithm of data condensation (Expressionist from GeneData, Basel, Switzerland). The results for each gene were expressed as the number of comparisons showing regulation in either direction. Pairwise comparisons were only considered if they were above an arbitrary cut-off factor of 1.3.

In order to select the most representative differentially expressed genes, two different selection strategies were applied. The first was based on a numerical criterion, selecting genes that were equally regulated (down or up) in ≧75% of the individual pairwise comparisons, and allowing ≦5 pairwise comparisons with the opposite tendency in no more than one individual (patient or HD). The second strategy was to make a selection from amongst all the genes relevant to MS pathophysiology. Here, the inventors used the mean value of the regulation in each patient after multiple pairwise comparisons. Regulation in the same direction was required in ≧7 out of 10 RRMS patients or ≧6 out of 8 PPMS patients, as compared to HD, allowing the opposite tendency in only one patient (up or down regulation). From these group of genes chosen after applying the first (numerical) or second (genes of interest) criterion, the inventors selected only those that showed a relative expression greater than 2 (up or down).

Quantitative Real-Time rtPCR

Total RNA was reversely transcribed to cDNA with random hexamers using the TaqMan® Reverse Transcription Reagents (Perkin Elmer, Foster City, Calif.), according to the manufacturer's instructions. Quantitative real-time rtPCR was performed on an ABI Prism® 7700 Sequence Detection System (Perkin Elmer) (Wandinger K P, Sturzebecher C S, Bielekova B, et al. Complex immunomodulatory effects of interferon-beta in multiple sclerosis include the upregulation of T helper 1-associated marker genes. Ann Neurol. 2001; 50:349-357).18 Primers and probe for CX₃CR1 were designed using Primer Express software (Perkin Elmer), except for 18S rRNA (Perkin Elmer): forward 5′-TGA CTG GCA GAT CCA GAG GTT-3′ (SEQ ID NO: 1); reverse 5′-TTC TGT CAC TGA TTC AGG GAA CTG-3′ (SEQ ID NO: 2); probe 5′-AGT CCA CGC CAG GCC TTC ACC A-3′ (SEQ ID NO: 3). The probe was labeled with 6-carboxy-fluorescein (FAM) as reporter dye and 6-carboxytetramethyl-rhodamine (TAMRA) as quencher dye. Different primer and probe concentrations (100 to 800 nM) were tested to optimize the PCR amplification. Thermal cycling conditions used were the following: 2 min at 50° C. and 95° C. for 10 min followed by 40 cycles of 95° C. for 15 sec and 60° C. for 1 min. The PCR reaction was carried out in a 50 μl final volume. All samples were run in duplicate.

Comparative threshold method (comparative Ct method) was used to quantify the results obtained by real-time rtPCR. The number of targets (mRNA) was normalized to the corresponding endogenous housekeeping gene (18S rRNA) for each sample to adjust for uncontrolled variability between samples.

### Flow Cytometric Analysis

mAbs to human CX₃CR1 (clone 2A9-1; rat IgG2b) were kindly provide by Dr. Imai from Kan Research Intitut (Kioto, Japan). PBMC from MS patients and healthy individuals (Table 1C) were stained as described previously.¹⁴ In brief, cells were first incubated with anti-CX₃CR1 for 20 min. at room temperature and then stained with APC-conjugated goat anti-rat IgG (Cedarlane, Canada). Afterwards, cells were incubated for 15 min with 1% rat serum and finally stained with anti-CD3 FITC/anti-CD56CD16 PE, anti-CD3/anti-CD4 or anti-CD3/anti-CD8 (all Abs from BD) for further 20 min. Samples were analysed by four-color flow cytometry on a FACScalibur, and 10,000 gated T cells or NK cells were acquired for each sample.

### Statistical Analysis

The Mann-Whitney-U-test was used for the calculation of p-values for inter-group comparisons of the microarray results, the rtPCR products and the cytometry data. The calculation was carried out using SPSS 10.0 software for Windows (SPSS, Chicago, Ill., USA). A p-value of <0.05 was regarded as significant.

TABLE 1
Characteristics of Patients and Healthy Donors
A) Patients and HD involved in the oligonucleotide-based DNA microarray study

| | Number | Female/Male | Age | Mean EDSS |
|---|---|---|---|---|
| HD | 12 | 6/6 | Mean: 29; range: 24-41 | — |
| RRMS | 10 | 8/2 | Mean: 29; range: 20-39 | Mean: 1; range: 0-1.5 |
| PPMS | 8 | 4/4 | Mean: 52; range: 38-66 | Mean: 5; range: 3-8 |

B) Patients and HD involved in the TaqMan analyses

| | Number | Female/Male | Age | Mean EDSS |
|---|---|---|---|---|
| HD | 28 | 16/12 | Mean: 31; range 24-48 | — |
| RRMS | 25 | 21/4 | Mean: 32; range 17-46 | Mean: 1; range: 0-4 |
| PPMS | 20 | 12/8 | Mean: 53; range 38-66 | Mean: 5; range: 1.5-7.5 |

C) Patients and HD involved in flow cytometry analyses

| | Number | Female/Male | Age (range) | EDSS (range) |
|---|---|---|---|---|
| HD | 19 | 14/14 | Mean: 30 (19-54) | — |
| RRMS | 19 | 16/3 | Mean: 36 (24-47) | Mean: 1 (0-4) |

D) Patients and HD involved in the analysis of clinical disease activity and CX₃CR1
expression

| | Number | Female/Male | Age (range) | EDSS (range) |
|---|---|---|---|---|
| HD | 28 | 14/14 | Mean: 29 (19-54) | — |
| RRMS | | | | |
| stable | 13 | 12/1 | Mean: 38 (27-46) | Mean: 1 (0-4) |
| active | 9 | 6/3 | Mean: 31 (24-37) | Mean: 1.3 (0-3.5) |

TABLE 2
Clinical characteristics and frequency of CX₃CR1⁺ NK
cell in RRMS patients with stable and active disease

| | | % | | Gd- | | Other |
|---|---|---|---|---|---|---|
| Patient | Sex/ | CX₃CR1⁺ | Acute | enhancing | | diseases/ |
| no. | age | NK cells | relapses | lesions | EDSS | therapies |
| 1 | F/46 | 0.0 | no | no | 0 | |
| 2 | F/33 | 0.0 | no | no | 0 | Respirat. |
| | | | | | | Infection |
| 3 | F/27 | 3.1 | no | no | 1.5 | Respirat. |
| | | | | | | Infection |
| 4 | F/44 | 0.0 | no | no | 2 | |
| 5 | F/30 | 0.0 | no | no | 0 | |
| 6 | F/41 | 0.0 | no | no | 1 | Herpes |
| | | | | | | labialis |
| 7 | F/43 | 2.2 | no | no | 1 | |
| 8 | F/39 | 0.0 | no | no | 4 | |
| 9 | F/45 | 0.0 | no | no | 0 | |
| 10 | F/34 | 0.0 | no | no | 0 | |
| 11 | F/34 | 37.5 | no | no | 0 | |
| 12 | F/46 | 6.6 | no | no | 2 | |
| 13 | M/35 | 0.0 | no | no | 2 | |
| 14 | M/33 | 11.8 | no | yes | 0 | |
| 15 | F/27 | 34.6 | 14 d after | yes | 1 | |
| | | | venipunct. | | | |
| 16 | F/24 | 17.6 | yes | yes | 2.5 | Steroids |
| 17 | F/28 | 33.1 | yes | nd | 1 | |
| 18 | F/36 | 23.6 | yes | nd | 2.5 | |
| 19 | M/32 | 23.6 | no | yes | 1 | |
| 20 | F/37 | 13.0 | yes | nd | 3.5 | |
| 21 | F/34 | 34.5 | no | yes | 0 | |
| 22 | M/28 | 0.0 | no | yes | 0 | Respirat. |
| | | | | | | Infection |

nd = not done

1. In vitro method for the diagnosis of multiple sclerosis (MS) in a patient, comprising
a) providing a biological sample from the patient to be diagnosed,
b) providing a biological sample from a healthy donor or a reference value indicative for a healthy donor, and
c) determining the level of expression of CX3CR1 gene or protein expression in said samples,
wherein a reduction of the relative expression of more than 30% compared to said healthy donor is indicative of MS in said patient.

2. In vitro method for the diagnosis of multiple sclerosis in a patient according to claim 1, characterised in that the relative expression is reduced by at least 2-fold, preferably 3-fold.

3. In vitro method for the diagnosis of multiple sclerosis in a patient according to claim 1, characterised in that said the biological samples are enriched for peripheral mononuclear cells (PBMC).

4. In vitro method for the diagnosis of multiple sclerosis in a patient according to claim 1, characterised in that said patient suffers from RRMS or PPMS.

5. In vitro method for the diagnosis of multiple sclerosis in a patient according to claim 4, characterised in that the gene expression is reduced by at least 30% and at least 40% in patients suffering from RRMS and PPMS, respectively.

6. In vitro method for the diagnosis of an acute and/or stable multiple sclerosis in a patient, comprising
a) providing a biological sample from the patient to be diagnosed containing NK cells, and
b) determining the CX3CR1 protein expression in said sample,
wherein an expression of CX3CR1 protein in more than 15% of the NK cells is indicative of acute MS in said patient.

7. In vitro method for the diagnosis of multiple sclerosis in a patient according to any of claim 6, wherein the biological sample is enriched for NK cells.

8. In vitro method for the diagnosis of multiple sclerosis in a patient according to claim 1, wherein determining said CX3CR1 gene or protein expression level comprises microarray analysis, real-time PCR and/or flow cytometry analysis.

9. In vitro method for the diagnosis of multiple sclerosis in a patient according to claim 1, wherein said biological sample is whole blood or serum and said CX3CR1 protein expression analysis comprises flow cytometry analysis.

10. In vitro method for the diagnosis of multiple sclerosis in a patient according to claim 1, wherein said method is performed at least one to four times a year.

11. A method of treatment for multiple sclerosis in a patient, comprising the steps of
a) performing the method according to claim 1, and
b) providing a suitable medication to a patient in need thereof.

12. Method of treatment for multiple sclerosis in a patient according to claim 11, wherein the said patient suffers from acute RRMS or PPMS.

13. Kit, comprising suitable materials and compounds for performing the method according to claim 1.

14. Kit according to claim 13, wherein said kit comprises materials, charts and compounds suitable for a point-of-care analysis.

15. Use of the expression of CX3CR1 for distinguishing between acute and stable MS, in particular RRMS and/or PPMS disease phase.


SEQUENCE LISTING (3 sequences)

SEQ ID NO: 1 - DNA, 21 bp, Homo sapiens
tgactggcagatccagaggtt

SEQ ID NO: 2 - DNA, 24 bp, Homo sapiens
ttctgtcactgattcagggaactg

SEQ ID NO: 3 - DNA, 22 bp, Homo sapiens
agtccacgccaggccttcacca